(19) United States
(12) Patent Application Publication
(10) Pub. No.: US 2016/0095816 A2
(43) Pub. Date: Apr. 7, 2016

(54) ARTICLE AND METHODS FOR ORAL SELF-ADMINISTRATION OF NICOTINE

(71) Applicant: Evan Grossman, Fort Lauderdale, FL (US)

(72) Inventor: Evan Grossman, Fort Lauderdale, FL (US)

(21) Appl. No.: 14/507,019

(22) Filed: Oct. 6, 2014

Related U.S. Application Data
(63) Continuation-in-part of application No. 13/459,070, filed on Apr. 27, 2012, now abandoned.

(51) Int. Cl. A61K 9/00; A61K 47/10; A61K 31/465; A24B 15/16; A24F 47/00

(52) U.S. Cl. CPC A61K 9/0092; A24B 15/16; A24F 47/00; A61K 31/465; A61K 47/10

(57) ABSTRACT

The invention discloses an article for swaddling in the mouth to deliver pharmacologically active substances, particularly nicotine, in combination with dyes or flavorings, across the buccal membranes when said article is swaddled in the mouth. The article comprises a non-edible substantially cylindrical core element, such as a toothpick, and the indicator element may be a bulb, trinket, insignia or other marking. In a preferred embodiment, the invention makes a social statement about the beneficial uses of nicotine. Methods for using the invention to mitigate sensations of nervousness and boredom are also disclosed.

### BACKGROUND OF THE INVENTION

1. Field of the Invention

The present invention relates to an article of manufacture, and methods for using the same, that comprises a non-edible substantially cylindrical core element, such as a toothpick, and an indicator element such as an insignia, wherein the article has been soaked in a mixture essentially comprising nicotine such that said nicotine is releaseably retained within the article until swaddled in the mouth of a user, whereupon said nicotine may diffuse cross the buccal membranes and enter the bloodstream of said user, wherein said article is neither a gum nor a lozenge, and wherein no plant material is ingested. Methods for using the article to mitigate sensations of nervousness and boredom are also disclosed.

2. Description of the Related Art

The oral delivery of nicotine into the human body via absorption across the buccal membranes and other capillaries in the oral cavity has been attempted in various ways, such as in cigarettes, chewing gums, sprays, lozenges, lollipops, candies, and bolus. Oral nicotine delivery mechanisms that do not involve tobacco products have been developed for a variety of reasons, such as:

For use as a tobacco substitute—see U.S. Pat. No. 5,417,229 to Summers et al. (“Organoleptic Bite Composition for Human Consumption”), U.S. Pat. No. 4,907,605 to Ray et al. (“Oral tabacco [sic] substitute”), U.S. Pat. No. 5,875,786 to Chase (“Cigar Substitute”), U.S. Pat. No. 5,824,334 to Stanley et al. (“Tobacco Substitute”), U.S. Pat. No. 5,048,544 to Mascarelli et al. (“Cigarette Substitute”), each of which is incorporated by reference herein;

For use as a smoking cessation aide—see U.S. Pat. No. 7,766,019 to Luzenberg, Jr. (“Porous Plastic Smoking Cessation Devices”), U.S. Pat. No. 7,766,018 to Roth et al. (“Device and Composition for Reducing the Incidence of Tobacco Smoking”), U.S. Pat. No. 7,156,109 to Sampson (“Smoking Cessation Oral Hygiene Device”), U.S. Pat. No. 6,583,160 to Smith et al. (“Nicotine Therapy Method and Oral Carrier for Assuaging Tobacco-Addiction”), U.S. Pat. No. 6,280,761 to Santus (“Nicotine Lozenge”), U.S. Pat. No. 5,783,207 to Stanley et al. (“Selectively Removable Nicotine Containing Dosage Form for Use in the Transmucosal Delivery of Nicotine”), each of which is incorporated by reference herein; and,

For use as a pharmaceutical—see U.S. Pat. No. 7,581,540 to Hale et al., (“Aerosol drug delivery device incorporating percussively activated heat packages”), U.S. Pat. No. 7,387,788 to Carrara et al. (Pharmaceutical Compositions of Nicotine and Methods of Use Thereof'), U.S. Pat. No. 5,147,654 to Place et al. (“Oral Osmotic Device for Delivering Nicotine”), U.S. Pat. No. 6,676,959 to Andersson et al. (“Nicotine-Containing Pharmaceutical Compositions Giving a Rapid Transmucosal Absorption”), and U.S. Pat. No. 6,586,449 to Walling (“Nicotine-Containing Controlled Release Composition and Method”), each of which is incorporated by reference herein.

Many of these previously developed nicotine products are complicated and costly. They are designed to release nicotine with a pharmacokinetic rate and dosage profile resembling that which is obtained from consuming a tobacco product. Prior art devices have incompletely explored the homeopathic and health-promoting activities of nicotine and have not been provided it in simple, cheap, low-dose forms which are ideal for harnessing the beneficial properties of nicotine, and which circumvent some of nicotine's addictive properties. Instead, these products have attempted to mimic the levels of nicotine found in addictive tobacco products because they were developed as smoking cessation aids or tobacco replacement products.

### Nicotine and Homeopathy

Nicotine is a heterocyclic alkaloid compound that exists in both free base and a salt forms. As a nitrogenous base, nicotine forms salts with most acids. Nicotine has many useful and beneficial properties, although when ingested by certain means, particularly cigarettes and bolus, it can cause intractable addiction. The term “nicotine” is defined broadly for the purposes of the present invention, wherein “nicotine” refers to the chemical substance named 3-(1-Methyl-2-pyrrolidinyl) pyridine or C[10]H[14]N[2], or 1-methyl-2-(3-pyridyl)pyrrolidine; B-pyridyl-alpha-N-methylpyrrolidine, and having a molecular weight of approximately 162, as well as any salt or complex thereof; and, “nicotine” also refers to any analogous compounds or molecules capable of agonizing or antagonizing nAChrs (nicotinic acetylcholine receptors) in the human body.

Nicotine is a potent modulator of nAChrs. Through these receptors, nicotine activates the presynaptic release of several neurotransmitters including acetylcholine, norepinephrine, serotonin and dopamine. Reports concerning nicotine's psychoactive effects are diverse and contradictory. Nicotine behaves as both a stimulant and a relaxant. Its stimulant activities are thought to be related to epinephrine release from the adrenal medulla, promoting feelings of sharpness and alertness in users. The precise mechanisms that contribute to users' alternative feelings of relaxation, calmness, and satisfaction (including reduced appetite for food) are unclear. Nicotine has a half life of 1-2 hours in the bloodstream and is metabolized in the liver. Glucuronidation and oxidative metabolism of nicotine to cotinine are both inhibited by menthol, which can increase nicotine half-life in vivo.

Nicotine has effects on bodily health which qualify it as a homeopathic compound. As previously mentioned, nicotine acts on the nicotinic acetylcholine receptors in various parts of the brain and central nervous systems. In small concentrations, it increases the activity of these receptors, which are sensitive to the concentration of nicotine in the blood. In large doses, it may be toxic or it may have reverse effects compared to low doses. Nicotine also has a variety of indirect effects on the release of neurotransmitters including dopamine and components of the reward pathways in the brain. Consequently, nicotine in various doses can produce feelings of euphoria and reduced anxiety. Nicotine also stimulates muscles tension, and at high doses, can cause spasms in skeletal muscle and respiratory paralysis.

Promising studies suggest that nicotine can promote muscle growth and wound healing due to its stimulatory effect on blood vessel growth (angiogenesis) and possible effects on stem cell growth. Nicotine also promotes increased heart rate, elevated blood pressure and respiration, and higher blood glucose levels through activation of the sympathetic nervous, ostensible via the epinephrine (adrenaline) pathway. Some studies report increased cognitive function after nicotine ingestion, but these results may be subjective depending on each individual user and circumstances of use. Additional reports exist suggesting that nicotine may alleviate symptoms of attention deficit hyperactive disorder (ADHD), attention deficit disorder (ADD), Tourette Syndrome, Schizophrenia, Parkinson's Disease, Alzheimer's Disease, anxiety, and depression. Nicotine is an extremely versatile pharmacological compound, with rather unpredictable yet predominantly beneficial effects in small doses.

Homeopathy, which dates back to the Nineteenth Century, is founded on the principles of pharmacology and biology. It is based on the observation that high doses of pharmacologically active substances cause symptoms when administered to healthy individuals, and that those same substances, when prepared in diluted concentrations, may relieve similar symptoms in conditions resulting from different etiologies. One of the earliest laws of pharmacology representing the homeopathic effect, the Arndt-Schultz law, states that every stimulus on a living cell elicits an activity, which is inversely proportional to the intensity of the stimulus (Martius F. Das Arndt-Schultz Gnindgesetz, Muench Med. Wschr., 1923, 70(31):1005-1006). This law was later restated by Ferdinand Hueppe as: for every substance, small doses stimulate, moderate doses inhibit, large doses kill.

Nicotine and several nicotine analogs are included in the official Homeopathic Monographs from the General Pharmacy of the Homeopathic Pharmacoepia of the United States. Said Homeopathic Monographs for nicotinum (nicotine) and nicotine analogs are incorporated by reference herein.

Nicotine potentially interacts with numerous attributes of the body to affect positive biological outcomes, such as reductions in psychological tension, increase in muscle tone, increased awareness and focus, decreased cravings for food, and others. Scientists have shown that nicotine promotes blood vessel growth, which can be beneficial to people with arterial diseases. Ironically, it is now thought that the high carcinogenicity of tobacco is caused by the combined actions of chemicals other than nicotine which initiate tumor growth, plus rapid blood vessel growth, fueled by nicotine, that empowers these tumors to become large and aggressive. Thus, the true culprits behind the carcinogenicity of cigarettes are the non-nicotine toxins which start tumor growth.

Research has conclusively demonstrated that the strongly addictive nature of cigarette smoking is related to the manner in which cigarette smoke introduces nicotine into the bloodstream. When a user inhales from a cigarette, a sudden burst of volatilized nicotine hits the lungs in a form that allows the nicotine to pass easily through the alveoli and into the bloodstream. The majority of the nicotine absorbed from each breath of smoke is consumed rapidly, trailing off slowly as the nicotine disperses and the smoker exhales. Various estimates have been made of the precise pharmacokinetics of smoked nicotine, and a general estimate is that upon inhaling from a lit cigarette, a burst of nicotine is quickly absorbed into the smoker's blood and reaches the brain within eight seconds. The peak of nicotine delivered into the bloodstream through smoking may be as high as 30-40 nanograms per milliliter of blood, and its peak absorption in the brain is reached within ten minutes (see Russell, M. A. H. in Nicotine Replacement: a Critical Evaluation; Pomerleau, O. F. and Pomerleau, C. S., Eds.; Alan R. Liss, Inc.: New York, 1988; pp 63-94).

Russell states that the optimal steady-state blood level for nicotine replacement is between 10-15 ng/mL, but that quick-rise effects are probably necessary for more complete relief from craving in the early stages of cigarette withdrawal. A rise in nicotine blood level of at least 10 ng/mL in 10 minutes is probably required to obtain postsynaptic effects at nicotinic cholinergic receptors in the central nervous system and at autonomic ganglia. This pharmacokinetic profile is distinct from absorption through the skin (as in a patch) or through the mouth (as in a gum). The blood level concentration peak produced by cigarettes is higher and sharper than steadier levels obtained from other types of delivery. One of the reasons gums and patches fail as smoking cessation aids is because they cannot mimic the rate of nicotine delivery offered by inhalation, although they are intended to do so.

One of the unpleasant features of orally administered nicotine and tobacco products is their tendency to induce nausea and gastric discomfort when swallowed. Currently marketed smoking cessation (SC) aids for oral administration such as nicotine gum or lozenges release a copious initial “burst” of nicotine, in part because of the inherent properties of these nicotine vehicles and in part by design, because such an initial burst of nicotine is essential to mimicking the effect that smoking a cigarette has upon nicotine levels in the bloodstream and thus is necessary to satiate the cigarette cravings of the user. The package labeling for these oral SC aids instructs users to chew intermittently, or to hold the saliva in the mouth for sufficient time to allow this initial burst of nicotine to dissolve across the buccal membranes and enter the bloodstream. This is unpleasant to the user, undesirable in a social setting, and difficult to achieve. When these instructions are not followed, the initial burst of nicotine an any attendant flavors is swallowed after which it is attenuated into the gastrointestinal tract, curtailing the absorbability of that portion of the nicotine from the SC aid and furthermore causing heartburn, nausea, and other unwanted ailments. For this reason, no effective SC aids are suitable for comfortable, relaxing use in a socially acceptable fashion while not inducing some level of irritation in the gastrointestinal tract.

Because SC aids must mimic the rate of absorption of nicotine from cigarette smoke into the blood stream in order to effectively replicate the psychoactive effects of cigarette smoking, thereby satisfying smokers' cravings, SC aids themselves are becoming increasingly addictive as they become increasingly effective. This is a moot consequence for those already addicted to cigarettes, but it renders SC aids undesirable for nonsmokers who wish to benefit from the many useful effects of nicotine without falling victim to the particularly addictive dose response curve of smoked tobacco.

Nicotine chewing gum and transdermal nicotine patches do not adequately reproduce the sharp initial rise in blood nicotine concentrations obtained by smoking cigarettes, although they provide comparable blood concentrations as achieved by smoking after about 30 minutes of chewing or absorption. In the case of gums which are used to counteract cravings for nicotine-containing products, a substantial portion of the nicotine from such gums may be swallowed because of slow absorption in the mouth. Nicotine gums and other chewable tobacco substitutes attempt to deliver too much nicotine through the mouth in too short a time, overwhelming the absorptive ability of the oral capillaries and inducing discomfort in the throat, stomach, and intestines after one or more milligrams of nicotine are swallowed.

Over-the-counter (OTC) tobacco replacement products are sold under brand names and private labels, and as generic products. They are approved for sale to persons 18 years of age and older. OTC nicotine replacement products include skin patches available as generics known as transdermal nicotine patches, as private-label products, and under the brand names Habitrol and Nicoderm. These patches are affixed to the skin, similar to an adhesive bandage, where they often lead to local skin irritation. Nicotine gum is available generically and under the brand name Nicorette. Nicotine lozenges are sold as generics, pharmacy medicaments, and brand name products such as Commit lozenges.

None of these tobacco replacement or smoking cessation products are intended to provide homeopathic levels of nicotine to the user. They are each intended to deliver the amounts of nicotine comparable to those experienced during tobacco use. Consequently, they counteract some of the health-promoting activities of nicotine. One way to represent this is to note that mildly toxic doses of nicotine, as well as local irritation of tissues, may occur through the manufacturer-recommended use of these products. The present invention delivers too small a dose of nicotine at too slow a rate to compare to these products.

Ariva and Stonewall are examples of smokeless dissolvable tobacco products, and are distinct from the present invention because the present invention is not a tobacco product and does not comprise tobacco. One piece of Arriva contains an average of 1.5 mg of nicotine, while one piece of Stonewall contains an average of 4 milligrams of nicotine. Other dissolvable tobacco products are marketed as Camel Dissolvables and Camel Sticks, which their manufacturer advertise as “made from tobacco that is finely ground. This tobacco is then combined with binders and non-characterizing flavorings that complement tobacco's natural taste.”

By definition, a lozenge dissolves in the mouth completely and all components are normally swallowed. A gum is chewed, said chewing being necessary to detach nicotine from its substrate. The present invention, therefore, does not qualify as a gum or lozenge. Instead, it is a nicotine delivery article intended to be swaddled in the mouth, with one end protruding from the mouth at all times.

In light of the above, there is a need for a nicotine delivery article and method that permits homeopathic and recreational self-administration of nicotine in a manner that is socially acceptable and non-fumigating, that requires no spitting or chewing, which does emit strong odors detectable by other people in the user's surroundings, and which does not mimic the pharmacokinetic properties of nicotine ingested through smoking or chewing of tobacco. There is also a need for a simple and inexpensive way to deliver homeopathic and recreational nicotine to those who desire it for its positive functions that help maintain healthy body function.

No admission is made that any reference, including any patent or patent document, cited in this specification constitutes prior art. The discussion of the references states what their authors assert, and the applicant reserves the right to challenge the accuracy and pertinency of any of the documents cited herein.

### SUMMARY OF THE INVENTION

The present invention satisfies the above stated needs. The present invention relies upon the slow, gradual absorption of nicotine within the oral cavity to provide homeopathic and recreational nicotine in a non-addictive, or less-addictive, dose. It is not a smoking cessation aide, nor does it mimic the pharmacokinetic profile for nicotine delivery posed by a smoking cessation aide. It is not a gum or a lozenge, and it contains no macroscopic plant matter. The present invention is expressly distinguished from smoking cessation aids and tobacco substitutes because it is not intended to mitigate the urge to smoke a cigarette or to chew tobacco, and it does not deliver nicotine with the requisite pharmacokinetic profile to replace tobacco products, but instead, it delivers nicotine in a slower, steadier fashion which is less prone to causing addiction and toxicity than when nicotine is obtained through direct consumption of tobacco products.

The present invention is more socially acceptable as a non-tobacco nicotine delivery article than a smokeless cigarette (i.e., a volatilized nicotine delivery article) because it does not release nicotine or flavorants into the air where others may be exposed to them. Because many consumers who support the recreational and homeopathic use of nicotine feel strongly about its promotion and its distinction from tobacco-based nicotine consumption, the present invention is also intended to identify its function and commercial origin to other people who observe the user swaddling the article in the mouth. For this purpose, the article includes an indicator element that may be a bulb (either painted or illuminated), a trinket, an insignia, or other indicia. Said indicator element is attached at or near the posterior end of the article so that it remains visible while the article is swaddled in a user's mouth, protruding out from the mouth during use.

In the present invention, nicotine is delivered to a user in a method and form of a substantially cylindrical non-edible solid, such as a toothpick, which has been pre-treated with a nicotine containing mixture such that nicotine is retained in or on said the article until the article is placed in the user's mouth, whereupon said nicotine is released by diffusion, assisted by the saliva, into the oral cavity where it is then absorbed through oral mucosa. Alternatively, the cylindrical core of the article may be inert while a distinct delivery element attached to the anterior end of the core contains the nicotine mixture. Advantageously, all elements of the article may be so treated with nicotine. The release of nicotine from the article into the oral cavity occurs at a different rate than that which occurs from either a smoking cessation aid or a tobacco substitute, permitting new and useful applications of nicotine for promotion of healthy body functions. The present invention also claims methods for alleviating the sensations of nervousness and boredom by delivering nicotine in small steady doses to a user through his or her oral tissues.

In a preferred embodiment, the base element comprises wood having been soaked in a mixture comprising essentially nicotine or nicotine analogs. Additional flavorants or chemical compositions are consistent with the invention and may also be included as described throughout this specification. Further embodiments may incorporate other pharmacologically active compounds in addition to, or in place of, nicotine. The article may comprise plastic, rubber, and any other non-digestible material. In some embodiments, the delivery element and/or the indicator element may be replaceable, such that alternative indicia could be attached and fresh cartridges of nicotine could be attached to the core element.

It is an object of the present invention to provide a means for oral self administration of nicotine that is suitable for comfortable, relaxing use in a socially acceptable fashion. It is a further object of the present invention to provide a means for oral self administration of nicotine to nonsmokers who wish to benefit from the many useful effects of nicotine without falling victim to the particularly addictive pharmacokinetics of nicotine absorption from smoked and chewed tobacco. It is still a further object of the present invention to deliver nicotine in a dose and at a rate that are small and slow, respectively, thereby limiting the potential to produce toxicity, gastric discomfort, or local irritation in the user. It is still a further object of the present invention to provide a socially acceptable means for oral self administration of nicotine which does not include tobacco or plant tissues and which does not require chewing, spitting, or swallowing of any plant, candy, filler, binder, or other solid material. Because all structural elements of the present invention are non-digestible and do not dissolve or change shape appreciably in the mouth of a user, the present invention is not a lozenge, nor a gum, nor a lollipop. The present invention provides a simple and inexpensive way to deliver homeopathic and recreational nicotine to those who desire it for its positive functions that help maintain healthy body function.

Each article of the present invention may contain a total quantity of between 0.1 and 5 mg of nicotine. The rate of diffusion of said nicotine into the user's oral cavity upon swaddling the article in the mouth may vary substantially depending on the properties of the base material and the actual quantity of nicotine present within said base at the time of introduction to the oral cavity of a user.

The term “use” or “using” of the present invention encompasses swaddling, sucking, gripping between the teeth, or otherwise orally positioning the article in order to extract nicotine through the oral cavity into the bloodstream, such that the posterior end of the article protrudes from the mouth during use. By using the present invention, a user may gain several benefits. First, the nicotine can promote healthy function of the body by its various actions on numerous receptors throughout the nervous system and other tissues. Second, the use of the present invention alleviates sensations of boredom experienced by an individual. Third, the use of the present invention alleviates sensations of nervousness experienced by an individual. Fourth, the use of the present invention provides a conspicuous social statement that the user is enjoying a homeopathic remedy comprising nicotine.

One of the features tobacco users enjoy about tobacco products is the organoleptic piquancy or “bite” which emanates from the inherent pungency of the chemical constituents of tobacco leaves. Paradoxically, this piquancy generates odors that non-tobacco users consider to be offensive and socially unacceptable. It is an object of the present invention to provide an article comprising essentially nicotine which may also include organoleptic agents to impart piquancy of taste to the user without including plant material that must be spit or swallowed. The organoleptic bite of the present invention need not be identical to that of tobacco found in nature.

According to the dictionary, “organoleptic” denotes: being, affecting, or relating to qualities that stimulate the sense organs, such as taste, color, odor, and feel, of a substance (as a food or drug). Thus, the invention contemplates the use of dyes and food colorings in addition to flavorants. “Organoleptic bite” is a term that first appears in the United States patent literature in U.S. Pat. No. 4,887,620 to describe the pepper-like aspect of tobacco extract. Organoleptic bite may refer to any sharp or pungent quality, pepper-like quality, or strongly bitter flavor that is perceived to be felt as well as tasted by the user. Organoleptic bite generally arises from phenolic compounds present within a substance such as an herb or spice. For example, piperine is the phenolic compound that imparts much of the organoleptic bite in black pepper, capsaicin in chili peppers, myristicin in nutmeg and mace, gallic acid in coffee, and cinnamaldehyde in cinnamon.

Organoleptic agents suitable for inclusion in the present invention and methods include, but are not limited to, each of the compounds described in the preceding paragraph as well as cola, menthol, methyl salicylate, biologically active vitamin or mineral preparations, other alkaloids, chicory, clover, cayenne pepper, ginger, dandelion, papaya, dock or sorrel, sunflower, calendria, nasturtium, mallow, rose hips, peppermint, spearmint, mint, aniseed, eucalyptus, carvone, anethole and any sharp, acrid, bitter, pungent, peppery, spicy, or aromatic flavorings; also vanillin, benzaldehyde, furanol, herb oils, spice oils, and citrus oils.

In an alkaline environment, e.g., in a pH above approximately 7.0, and in the presence of an aqueous medium, such as saliva within the oral cavity, nicotine salts react to form nicotine base. Because saliva normally has a somewhat acidic pH, the incorporation of an alkaline salt into the dosage-form of the present invention may be desirable to buffer the pH and facilitate formation of nicotine base. Preferred alkaline salts include sodium carbonate, sodium bicarbonate, potassium carbonate, potassium bicarbonate, trisodiun phosphate, disodium hydrogen phosphate, sodium oxylate, sodium succinate, sodium citrate, trimethamine and sodium salicylate.

The forgoing summary has outlined some features consistent with the present invention in order that the following detailed description thereof may be better understood, and in order that the present contribution to the art may be better appreciated. The present invention is not limited in its application, details, or components merely to those set forth in the following description and illustrations. Devices consistent with the present invention are capable of other embodiments. Also, the phraseology and terminology employed herein are for the purpose of description and should not be regarded as limiting unless explicitly stated as such. Those skilled in the art will appreciate that the conception upon which this disclosure is based may readily be utilized as a basis for designing of other structures, methods, and systems for carrying out the several purposes of the present invention. It is important, therefore, that the claims be regarded as including such equivalent constructions insofar as they do not depart from the spirit and scope of the devices consistent with the present invention.

### BRIEF DESCRIPTION OF THE DRAWINGS

FIG. 1 is a perspective view of a first embodiment of the article.

FIG. 2 is a perspective view of a second embodiment of the article comprising a delivery element and an indicator element.

FIG. 3 is a perspective view of a third embodiment of the article comprising an alternative delivery article and a trinket as the indicator element.

### DETAILED DESCRIPTION OF THE INVENTION

Sources of nicotine for the manufacture and use of the present invention include, but are not limited to, nicotine polacrilex, nicotine bitartrate, and nicotine salts. “Nicotine salt” refers to any mono- or bis-Pharmaceutically acceptable addition acid salt or metal salt of nicotine. Some nicotine salts suitable for incorporation into the present invention include nicotine hydrochloride, nicotine dihydrochloride, nicotine sulfate, nicotine citrate, nicotine monotartrate, nicotine bitartrate, nicotine zinc chloride monohydrate and nicotine salicylate. Prior to incorporation into the present invention, nicotine stock may be further processed or purified by distillation, dilution, filtration or other means.

Nicotine may be sorbed or similarly impregnated into the structural elements of the article by various means including immersion in liquid, immersion in a mixture comprising propylene glycol, exposure to nicotine vapors or by impregnation with a nicotine-containing solution. If desired, a pharmacologically acceptable anti-oxidant such as propyl gallate, butylated hydroxyanisole or butylated hydroxytolene may be added to impregnated nicotine to prevent oxidation.

The article of the present invention is not contained completely within the mouth of its user, but rather, one end protrudes from the mouth during use. In this regard, the present invention is not a substitute for chewing tobacco. Furthermore, the article does not need to be chewed in order to release sufficient nicotine into the oral cavity to achieve its function(s), and therefore it is not a substitute for nicotine gum. And, the base portion of the article does not appreciably dissolve in the mouth of a user, and therefore the article is not a lozenge. Also, the article does not contain a dissolvable candy or other edible material which would classify it as a lollipop.

The manufacture of each article of the present invention may be performed via various methods sufficient to embed, deposit, saturate, or otherwise introduce nicotine into or onto the material of the base element. In at least one embodiment, this is achieved by first dissolving nicotine salts in a volatile solvent such as ethyl alcohol, then exposing the base element to this solution as the solvent evaporates, either with or without the addition of heat. It is understood that nicotine salts exhibit good stability and shelf-life while nicotine base exhibits excellent absorption through oral mucosal membranes, and various strategies may be employed together or alternatively in the manufacture of the article. Nicotine salts may be formed with nicotine and an organic acid such as citric acid, acetic acid, tartaric acid, lactic acid, and oxalic acid. Nicotine may also be introduced as a chemical base. Nicotine is exuded from the present invention into the oral cavity at a relatively slow, constant rate. In at least one embodiment, this rate is defined as less than 30% per hour.

In a preferred embodiment of the present invention, toothpicks are placed into a nicotine solution (the term “solution” equivalent here to “mixture”) for 48 hours. The nicotine solution comprises nicotine dissolved to maximum solubility in a solvent, where the solvent may be water, alcohol, petroleum derivatives such as Propylene Glycol (PG), Polyethylene Glycol (PEG), and Ethylene Glycol (EG), herbal extracts, oils, and nontoxic organic solvents. Alternatively, the nicotine solution may contain nicotinum at homeopathic dilutions, such as the 6× dilution called for in the official Homeopathic Monograph for nicotinum in the General Pharmacy of the Homeopathic Pharmacoepia of the United States. The 48 hour incubation of the article, or of the delivery element of the article, in the nicotine solution may be performed at room temperature, or it may be performed at higher temperatures to increase the solubility of nicotine and other compounds in the solution, or at lower temperatures to slow the degradation of nicotine and other compounds in the solution. After the 48 hour incubation period, the solvent may be poured off, or evaporated away. Other embodiments involve longer and shorter incubation periods. A final drying step may be performed, as in a vacuum chamber. The manufacturing methods, solvents, and time periods applied will vary as appropriate for the particular chemical compounds in the solution. For example, a solution containing menthol or other easily volatilized compounds is advantageously incubated with the article at a temperature consistently below room temperature.

Several additives may also be present in the nicotine solution. Organoleptic agents may be added to taste. In one embodiment, commercially available cinnamon oil is added at the rate of two ounces per standard cup of water or alcohol. Alkaline buffers may be added to regulate the formation of nicotine base. In one embodiment, sodium bicarbonate is added at a rate of 1 mg per 100 mls. The present invention contemplates the addition of anti-oxidants and preservatives to stabilize nicotine molecules and ions in the solution during incubation, in the base material after drying, or in the oral cavity during use. Such additives are differentially incorporated as appropriate for the various flavorants, dyes, or pharmacologically active substances in the solution used in different varieties or “blends” of the invention.

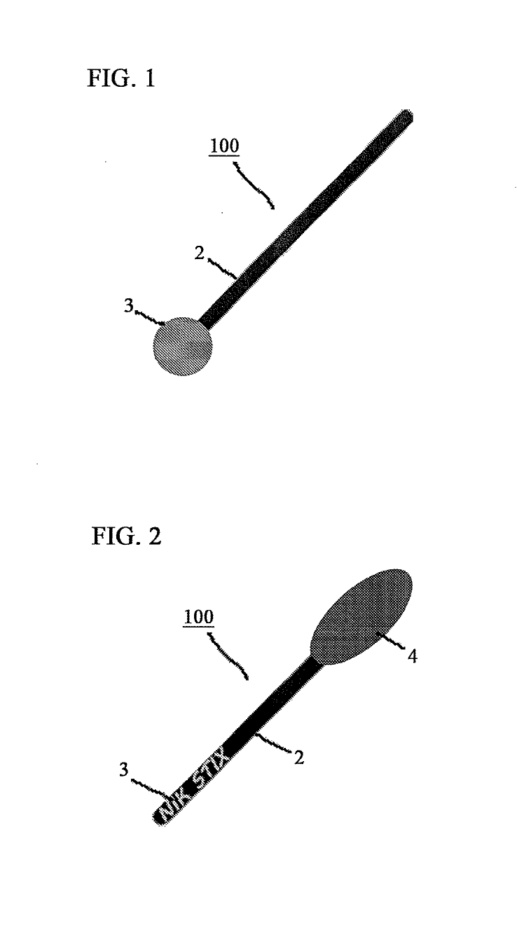

In a first embodiment of the article 100 illustrated in FIG. 1, the nicotine is perfused in the core element 2 which comprises wood and which has an indicator element 3 attached to its posterior terminus. The indicator element 3 is a bulb, either painted on a knob protruding from the core element or comprising an illuminating element such as an LED light affixed to the posterior end of the core element.

In a second embodiment of the article 100 illustrated in FIG. 2, a delivery element 4 is attached to the anterior end of the core element 2, the delivery element 4 comprising an indigestible, non-dissolving porous plastic material somewhat spherical in shape, suitable for absorbing a nicotine solution. The indicator element 3 is an insignia painted on the core element 2 near the posterior end or comprising foil lettering glued or otherwise affixed to the core element. The insignia may be a trademark such as Nik-Stix, Nix-Stix, Nik-Stik, or Nix-Stik.

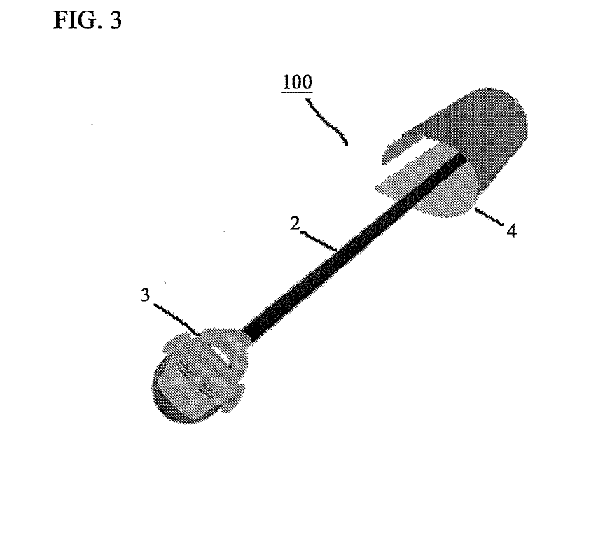

In a third embodiment of the article 100 illustrated in FIG. 3, the delivery element 4 is a roll of paper, fabric, or other absorptive material wrapped around the anterior end of the core element 2 and glued in place. The delivery element 4 is shown in a partially exploded or unwrapped state, but in the final construction the wrapping would be tightly wound around the core element 2. Alternatively, the delivery element 4 may be tied, clamped, or otherwise fastened in place. The indicator element 3 is a trinket. Trinket is a term that may comprise any decoration including but not limited to a carving, a cast object (e.g., a molded plastic), a gem or fake gem, a symbolic emblem, or the like. The trinket may bear a likeness to one or more celebrities, sports mascots, politicians, geographic landmarks, popular cultural icons, logos of sports teams, companies, or franchises, and the like. In FIG. 3, the trinket is a figure depicting the bust of a political personality

In any embodiment, a delivery element or the core element, or both, may be perfused with a nicotine solution or other flavorants, dyes, and pharmacologically active compounds. Multiple elements of the invention may be perfused with different solutions to achieve different flavoring results when swaddled in the mouth of the user. In a particular embodiment, a core element of the invention is perfused with nicotine while a delivery element is perfused with a cinnamon flavorant. In further embodiments, the delivery element may be a compound element comprising various materials perfused with different solutions. For example, the delivery element may be a dual-wrapper wherein one portion of the delivery element is a wrapper perfused with a first solution and the other portion of the delivery element is a second wrapper perfused with a second solution. These two wrappers could be layered, one on top of the other, or they could be positioned side-by-side along the length of the core element near the anterior end.

The dimensions of the article may vary within the structural scheme of the invention. The core element may have a circumference of between 0.5 and 25 millimeters. The delivery article is presented in a distinct geometrical shape, such as a sphere, cube, or multifaceted object. The outer length and width of the delivery article may vary between several millimeters to an inch. When the delivery article is a thin sheet of paper or a fabric wrapped around the core element, it may be wrapped one or multiple times around the circumference of the core element, and it is positioned substantially upon the anterior portion of the core element.

The article is used by a person in the following manner. A toothpick prepared in the above fashion is removed from a packaging or a temporary container and inserted into the mouth. The posterior end will protrude from the mouth, while anywhere from 50-90% of the remainder of the article will be wholly contained within the mouth. The article may be held in place between the teeth, cradled between check and gums, or rested on the tongue, among other configurations. Preferably, the user will alternate between several configurations to avoid irritating the skin in any one area of the mouth. The saliva provides a solvent into which the nicotine and other compounds dissolve from the article. Most of the nicotine enters the body through the capillaries of the oral tissues and not through the digestive tract. Importantly, the user is never required to spit or to hold the saliva in the mouth.

Boredom and nervousness are two human sensations that can be mitigated and alleviated by use of the present invention without substantial toxicity or addictive potential due to the slow, steady rate of nicotine absorption by the bloodstream from this article. In one preferred embodiment, a user recognizes or anticipates a sensation of either boredom or nervousness, and subsequently inserts an article consistent with the present invention into his or her mouth. The article is retained substantially within the mouth, and the user need not follow any designated method of chewing or swallowing-avoidance in order to reap the intended delivery of nicotine. After a period of 5-30 minutes, the user experiences alleviation, mitigation, or reduction of said sensation of either boredom or nervousness due to the quantity and rate of nicotine absorption through the oral tissues from the inserted article.

A method for alleviating nervousness according to the present invention comprises the steps of exposing an article comprising a toothpick, or any equivalent single-piece non-edible substantially cylindrical core element, to a solution comprising nicotine such that nicotine is absorbed into the core element and/or a delivery element, or otherwise releaseably retained therein, and then providing said article to one or more users to swaddle in the mouth, where after said nicotine is transferred from said article into the mouth of each user to cross the buccal membranes and oral capillaries into the bloodstream where the nicotine reaches the brain and modulates neurotransmission mitigate nervousness. Alternatively, the same method can be used to mitigate boredom.

Shelf life of the product may be enhanced using a material such as Vegetable Glycerin (VG). Too little VG (or too much water) and the nicotine and water can evaporate from the wood. Proper preferred moisture and consistency of wood is obtained by using VG and distilled water at the proper discovered levels. Too little VG (or too much water) and the wood can dry out through evaporation. Hygroscopic material such as VG mixes well with water. Hygroscopic material such as VG allows for diffusion of nicotine solution out of the wood and into the oral cavity due to the presence of water in saliva. Too little VG (or too much water) and the wood can become weak, brittle or too rubbery. Too much VG and the wood cannot absorb the solution since the VG is too viscous on its own to enter into the pores of the wood. Vacuum infusion is the preferred method to open up the wood pores and push the viscous solution into the wood pores. VG is considered as GRAS by the FDA. VG is used as a more organic material for skin lotions and hair products. While being more conscious of skin irritation problems caused by using Propylene Glycol, VG is the preferred hygroscopic material. VG is used as an artificial sweetener and a drug additive. VG makes nicotine more palatable to the user. Mint, Cinnamon, and tobacco flavorings may be added. Mint and tobacco can be added together to mimic the taste of common smokeless tobacco products.

Certain embodiments of the present invention may include the following representative formulations:

| | |
|---|---|
| 3% | Pure Nicotine Extract (≧98% purity) |
| 59.5% | USP Kosher Vegetable Glycerin |
| 37.5% | Distilled Water |
| 3.2% | Pure Nicotine Extract (≧98% purity) |
| 61.8% | USP Kosher Vegetable Glycerin |
| 33.33% | Distilled Water |
| 3.9% | Pure Nicotine Extract (≧98% purity) |
| 61.1% | USP Kosher Vegetable Glycerin |
| 35% | Distilled Water |

The following Examples provide specific embodiments which fall within the scope of the present invention.

### EXAMPLE 1

### Original Flavor

a. ≧98% pure organic Nicotine Extract makes up 0.5% to 10% of solution.
b. ≧98% Vegetable Glycerin (Glycerin/Glycerol) makes up 45% to 80% of solution.
c. 100% H2O (distilled water preferred) makes up 20% to 50% of solution.

### EXAMPLE 2

### Mint Flavored

a. ≧98% pure organic Nicotine Extract makes up 0.5% to 10% of solution.
b. ≧98% Vegetable Glycerin (Glycerin/Glycerol) makes up 45% to 80% of solution.
c. 100% H2O (distilled water preferred) makes up 20% to 50% of solution.
d. Mint, spearmint, peppermint oils/extracts make up 1% to 10%

### EXAMPLE 3

### Cinnamon Flavored

a. ≧98% pure organic Nicotine Extract makes up 0.5% to 10% of solution.
b. ≧98% Vegetable Glycerin (Glycerin/Glycerol) makes up 45% to 80% of solution.
c. 100% H2O (distilled water preferred) makes up 20% to 50% of solution.
d. Cinnamon oil/extracts make up 1% to 10%

### EXAMPLE 4

### Tobacco Flavored

a. ≧98% pure organic Nicotine Extract makes up 0.5% to 10% of solution.
b. ≧98% Vegetable Glycerin (Glycerin/Glycerol) makes up 45% to 80% of solution.
c. 100% H2O (distilled water preferred) makes up 20% to 50% of solution.
d. Tobacco flavorings/oils/extracts derived from natural and or artificial flavorings that may or may not be derived directly from tobacco make up 1% to 10%

Embodiments of the present invention also include methods of manufacture. One such method includes placing toothpicks vertically in a mason jar and adding appropriate amount of solution (1 ml for every 10 toothpicks). We are using the 2^{nd} formula listed above with 32 mg/ml of nicotine (in a solution containing 33.33% distilled water).

Next, a top is placed over the jar and the jars are placed in a large vacuum machine. After about 15 seconds, the jars seal and the solution starts to penetrate and run up the toothpicks. The jars are then placed on a rack for 2-7 days until the toothpicks have absorbed all the moisture and solution.

Some embodiments of the present invention may require special packaging as the finished toothpicks are slightly moist. A cardboard sleeve with an interior that is lined with an FDA approved film for food and drugs may be employed as part of the present invention. The film is to keep the nicotine from leeching out of the toothpick and into the paperboard/cardboard packs and also to keep the Pix fresh and moist over time (enhances shelf life). In certain instances, the nicotine solution may leech through the packaging. As such, the present invention contemplates the use of a polybag sleeve as part of the overall packaging of the product.

The toothpicks sit inside a polybag sleeve made of FDA approved polypropylene with the top end open for easy access by the consumer. This also serves as an additional level of freshness and enhancement of shelf life.

The packaging may also comprise shrinkwrap, shrinkband or overwrap. Again this enhances shelf life by holding moisture inside the package and inside the pix. Freshness is enhanced. Also this services as a barrier between the product and the outside environment. It also serves as a tamper proof seal. If the wrap is opened, the product has been tampered with.

It should be emphasized that the above described embodiments of the present invention exemplify some, but not all, possible implementations of the present invention and have been set forth in order to provide a clear understanding of its qualities. Variations and modifications may be made to the above-described embodiments of the cover of the present invention without departing from the spirit and principles of the invention. All such modifications and variations are intended to be included herein.

What is claimed is:

1. An article to be swaddled in a mouth of a person for oral self-administration of nicotine, the article comprising:
a core element comprising an absorbent material having an elongate shape, said core element having been soaked in a solution comprising a nicotine solution, wherein said nicotine solution comprises nicotine extract, vegetable glycerin, and distilled water; and
wherein said absorbent material of said core element is indigestible, such that while being swaddled in the mouth the core element retains said shape and is not digested by the digestive secretions of the human organism.

2. The article of claim 1, wherein the nicotine solution further comprises at least one material selected from the group consisting of flavorants, dyes, organoleptic agents, preservatives, solvents, pharmacologically active agents, and buffering agents.

3. The article of claim 1, wherein the nicotine solution includes a buffering agent that is an alkaline salt.

4. The article of claim 1, wherein the nicotine extract is present in the nicotine solution in a concentration of 0.5 to 10%.

5. The article of claim 4, wherein the vegetable glycerin is present in the nicotine solution in a concentration of 45 to 85%.

6. The article of claim 5, wherein the distilled water is present in the nicotine solution in a concentration of 20 to 50%.

7. The article of claim 6, wherein the nicotine solution further comprises at least one material selected from the group consisting of flavorants, dyes, organoleptic agents, preservatives, solvents, pharmacologically active substances, and buffering agents.

8. The article of claim 1, wherein the article comprises between 0.1 and 5 mg of nicotine.

9. The article of claim 1, wherein the article has been soaked in the nicotine solution for a period of time exceeding 24 hours.

10. The article of claim 1, wherein the absorbent material comprises at least one material selected from the group consisting of wood, plastic, and rubber.

11. The article of claim 1, wherein said core element is a toothpick.

12. A method of making a non-edible device for oral delivery of nicotine into a human body comprising:
providing a non-edible element comprising an absorbent material;
placing the non-edible element into a container;
contacting the non-edible element with a nicotine solution comprising nicotine extract, vegetable glycerin, and distilled water by placing the nicotine solution into the container with the non-edible element; and
allowing the non-edible element to remain in contact with the nicotine solution in the container for at least 24 hours.

13. The method of claim 12, further comprising the step of applying vacuum pressure to the container containing the non-edible element and the nicotine solution.

14.-20. (canceled)

21. The method of claim 12, wherein an amount of the nicotine solution placed into the container is about 1 ml for every 10 non-edible elements placed into the container.

22. The method of claim 12, further comprising allowing the non-edible elements to remain in contact with the nicotine solution for at least about 48 hours.

23. The method of claim 12, wherein the non-edible elements are placed in the container vertically.

24. The article of claim 1, wherein the vegetable glycerin is present in the nicotine solution in a concentration of 45% to 85%.

25. The article of claim 1, wherein the distilled water is present in the nicotine solution in a concentration of 20% to 50%.

26. The article of claim 1, wherein a release rate of nicotine from the core element upon swaddling in the mouth is less than 30% of the nicotine per hour.

27. The article of claim 1, further comprising a delivery element coupled to an anterior portion of the core element, wherein the delivery element is inedible and non-dissolving.

28. The article of claim 27, wherein the delivery element has the nicotine solution impregnated therein.

29. The article of claim 27, wherein the delivery element is pre-treated with a solution different than the nicotine solution, wherein the solution includes at least one of nicotine extract, flavorants, dyes, organoleptic agents, preservatives, solvents, pharmacologically active substances, and buffering agents.

30. The article of claim 27, wherein the delivery element is a bulb coupled to an anterior end of the core element.

31. The article of claim 30, wherein the bulb is spherical in shape.

32. The article of claim 27, wherein the delivery element is a fabric or paper wrapped one or more times around the anterior portion of the core element and secured thereto.

33. The article of claim 27, wherein the delivery element further comprises multiple delivery elements having different solutions impregnated therein.

34. The article of claim 1, further comprising an indicator element coupled to a posterior portion of the core element such that the indicator element protrudes from the mouth during swaddling.

35. The article of claim 1, wherein the nicotine extract is present in the nicotine solution in a concentration of 3% to 3.9%.

36. The article of claim 1, wherein the vegetable glycerin is present in the nicotine solution in a concentration of 59.5% to 61.8%.

37. The article of claim 1, wherein the distilled water is present in the nicotine solution in a concentration of 33.3% to 37.5%.

38. The article of claim 1, wherein the nicotine extract is present in the nicotine solution in a concentration of 3% to 3.9%, the vegetable glycerin is present in the nicotine solution in a concentration of 59.5% to 61.8%, and the distilled water is present in the nicotine solution in a concentration of 33.3% to 37.5%.

39. The article of claim 1, wherein the nicotine solution further comprises a buffering agent that is an alkaline salt and a material selected from the group consisting of flavorants, dyes, organoleptic agents, preservatives, solvents, pharmacologically active substances, and other buffering agents.

40. A nicotine product comprising:
the article of claim 1;
a cardboard sleeve having a film applied to an interior surface of the cardboard sleeve, wherein the article is disposed therein; and
a polybag sleeve disposed about the cardboard sleeve.

41. An article for self-administration of homeopathic amounts of nicotine to the body, the article comprising:
an elongate and porous core element pre-soaked in a nicotine solution to deposit nicotine therein, the nicotine solution including a nicotine extract and a solvent; and
wherein the core element is non-edible and is configured to release nicotine upon swaddling the article in a mouth of a user.

42. The article of claim 41, wherein the solvent further includes at least one at least one material selected from the group consisting of water, alcohol, petroleum derivatives, herbal extracts, oils, and non-toxic organic solvents.

43. The article of claim 41, wherein the solvent further includes at least one of the members of a group consisting of water, propylene glycol, polyethylene glycol, ethylene glycol, and vegetable glycerin.

44. The article of claim 41, wherein the solvent comprises vegetable glycerine and distilled water.
